(19) United States
(12) Patent Application Publication
(10) Pub. No.: US 2022/0168455 A1
(43) Pub. Date: Jun. 2, 2022

(54) HYPOCHLOROUS FOGGING OR MISTING APPARATUS AND METHODS

(71) Applicants: James Hirsch, Santa Barbara, CA (US); Shaun Valentine, Fort Thomas, KY (US); Brian Halleck, Santa Barbara, CA (US)

(72) Inventors: James Hirsch, Santa Barbara, CA (US); Shaun Valentine, Fort Thomas, KY (US); Brian Halleck, Santa Barbara, CA (US)

(21) Appl. No.: 17/675,841

(22) Filed: Feb. 18, 2022

Related U.S. Application Data
(63) Continuation-in-part of application No. 17/393,018, filed on Aug. 3, 2021.
(60) Provisional application No. 63/034,878, filed on Jun. 4, 2020.

(51) Int. Cl. A61L 2/22; B05B 7/24

(52) U.S. Cl. CPC A61L 2/22; B05B 7/2491; A61L 2202/14; A61L 2202/16; A61L 2202/15

(57) ABSTRACT

A disinfecting device is easy to use and can be easily mounted or removably connected in a dental operatory or other similar environment. The device can include a removable solution chamber attached to a base unit. The solution chamber can be a refillable chamber or a disposable chamber. The base unit can receive pressurized air, typically from a standard connection present in the dental operatory, or a dental chair, or the like. The pressurized air can be used to deliver a mist or an electrostatic fog of the solution in the solution chamber out of a nozzle. The solution bottle can be directly attached to the nozzle/sprayer device, with an air hose extending therefrom. In use, the user can turn on the system and use an activation/deactivation mechanism to deliver the atomized mist or fog to all surfaces in the room.

### BACKGROUND OF THE INVENTION

### 1. Field of the Invention

Embodiments of the invention relate generally to disinfection apparatus and methods. More particularly, embodiments of the invention relate to apparatus and methods for disinfecting dental operatories using a fog or aerosol mist.

### 2. Description of Prior Art and Related Information

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

Dental operatories are typically rooms in which a dental specialist may perform dental procedures on a patient. During procedures, droplets may be expelled from a patient's mouth via delivery of air or water, for example. Such droplets of blood, saliva, tissue, or the like, from the patient may contain potentially containing infectious pathogens. Thus, there is a need to disinfect such dental operatories between patients.

Typical methods include spraying down all surfaces, often with a hand-held spray bottle, or prewetted disinfecting wipes. After a predetermined time, the user then has to wipe down the surfaces to dry. Such a process can take 30 minutes of longer, during which time the operatory is not available for patient care.

In view of the foregoing, there is a need for improved methods and apparatus for disinfecting a dental operatory between patients.

### BRIEF DESCRIPTION OF THE DRAWINGS

Some embodiments of the present invention are illustrated as an example and are not limited by the figures of the accompanying drawings, in which like references may indicate similar elements.

FIG. 1 illustrates a side view of a disinfecting device according to an exemplary embodiment of the present invention;

FIG. 2 illustrates a detailed view of a sprayer unit, with the solution bottle removed, of the disinfecting device of FIG. 1;

FIG. 3 illustrates a detailed view of the sprayer unit, with the solution bottle disconnected from a sprayer unit bottle mount, of the disinfecting device of FIG. 1;

FIG. 4 illustrates a detailed perspective view of the sprayer unit of the disinfecting device of FIG. 1;

FIG. 5 illustrates a detailed perspective view of an air supply connector for the disinfecting device of FIG. 1;

FIG. 6 illustrates a handheld disinfecting device according to an exemplary embodiment of the present invention, in use in an operatory;

FIG. 7 illustrates perspective view of a portable disinfecting device according to an exemplary embodiment of the present invention, in use in an operatory;

FIG. 8 illustrates fixed mount disinfecting device, mountable in an operatory, according to an exemplary embodiment of the present invention;

FIG. 9 illustrates a wall mount disinfecting device, mountable in an operatory, according to an exemplary embodiment of the present invention;

FIG. 10 illustrates a user using the handheld disinfecting device of FIG. 6, using an optional pop-up lighting apparatus, according to an exemplary embodiment of the present invention;

FIG. 11 illustrates how disinfecting devices may be placed in a room to provide a disinfecting mist without user intervention, according to an exemplary embodiment of the present invention;

FIG. 12 illustrates an ability of the disinfecting devices of the present invention to communicate with a computing device to provide fogging data, for example; and

FIG. 13 illustrates an ability to provide one or more countdown timers to alert a user after an appropriate time has passed after applying a disinfecting mist.

Unless otherwise indicated illustrations in the figures are not necessarily drawn to scale.

The invention and its various embodiments can now be better understood by turning to the following detailed description wherein illustrated embodiments are described. It is to be expressly understood that the illustrated embodiments are set forth as examples and not by way of limitations on the invention as ultimately defined in the claims.

### DETAILED DESCRIPTION OF THE PREFERRED EMBODIMENTS AND BEST MODE OF INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention and is not intended to limit the invention to the specific embodiments illustrated by the figures or description below.

As is well known to those skilled in the art, many careful considerations and compromises typically must be made when designing for the optimal configuration of a commercial implementation of any system, and in particular, the embodiments of the present invention. A commercial implementation in accordance with the spirit and teachings of the present invention may be configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

Broadly, embodiments of the present invention provide a disinfecting device that is easy to use and can be easily mounted or otherwise connected for use in a dental operatory or other similar environment. The device can include a removable solution chamber attached to a base unit. The solution chamber can be a refillable chamber or a disposable chamber. The base unit can receive pressurized air, typically from a standard connection present in the dental operatory, or a dental chair, or the like. The pressurized air can be used to deliver a mist or an electrostatic fog of the solution in the solution chamber out of a nozzle. The solution bottle can attach directly to a handheld sprayer/nozzle. In other embodiments, a hose can connect the base unit with the nozzle, where the hose can be coiled, retractable, or the like, to permit the nozzle to reach the appropriate surfaces in the room to be disinfected. In use, the user can turn on the system and use a trigger on the nozzle to deliver the atomized mist or fog to all surfaces in the room. After a predetermined period of time, often less than 10 minutes, the operatory is ready for the next patient, without a need to wipe down the surfaces, wait for drying, and the like.

The device can include a built in timer so that, once the mist is applied, a button can be depressed on the hand unit and/or on the base unit to begin a timer that can provide an alert when the room is disinfected for the next patient. The device may also have wireless or wired communication ability to send the alert to a notification application on computer or smart device. In some embodiments, the device can link with a mobile computing device so that, once the mist is applied, the mobile computing device can detect the location (such as which operatory room) that was treated and begin a countdown to signify when the room is ready for occupancy.

The device may also have wireless or wired communication ability, where disinfection data may be saved to a database. Such data can include room disinfected, time disinfected, amount of disinfectant used, concentration of disinfectant, pH of the disinfectant, temperature and humidity levels of the room, and the like. This data allows the user to prove efficacy by allowing calculation of appropriate evaporation rates based upon temperature and humidity. The device's handpiece can include gyroscopes, accelerometers and/or GPS location devices to provide additional data regarding the application of disinfecting agent throughout the room.

Accordingly, in some embodiments, the device can include a sensor for detecting the concentration of the disinfecting agent and/or the pH of the disinfecting agent. The device can include a readout of this information or could simply provide an alert if any reading is outside of a predetermined range.

In one embodiment, the disinfecting agent can be a hypochlorous solution or a stabilized hypochlorous solution. In some embodiments, the hypochlorous solution can have a concentration from about 50 to about 400 ppm, typically from about 100 to about 200 ppm. The concentration may be without our outside of these typical ranges, depending on the particular application. Of course, the device of the present invention may be used with any disinfection agent that can be delivered via a spray, fog, mist or the like. The solution can be a commercially available solution, where the user can simply purchase and exchange solution containers as needed. The device can include a sensor to alert a user when the solution is low in the container. In other embodiments, the device can include an electrolysis unit, where the user places a chloride salt, such as sodium chloride, and water in the container and the device can automatically generate hypochlorous at the desired concentration. Such electrolysis devices are known in the art. The sensor for detecting the concentration of the disinfecting agent may be used to ensure the appropriate concentration of hypochlorous is generated and being used.

The device can also include a sensor to measure temperature and/or humidity in the room being disinfected. The measurement of temperature and humidity can be used to determine the disinfection time and how long the timer will wait until indicating the room is ready for the next patient. Such temperature and humidity data may also be stored in the database, along with the device-generated wait time for disinfection.

The misting/fogging handpiece body can be ergonomically shaped to be held in one hand (either left or right). The handpiece can include an activation/deactivation capability. The handpiece can include an aerosolizing tip or, in other embodiments, an electrostatic aerosolizing tip. The handpiece can include pressure regulating capabilities to account for the differing pressure level encountered in different operating environments. In some embodiments, a bottle holding disinfecting solution may attach directly to the handpiece. In other embodiments, a connecting tube can provide a connection between a first part of the device, housing the disinfecting solution, and a second part of the device, which delivers the mist or fog.

A software application can provide history on all captured date elements and reports that can be used to prove usage during an audit. The software application can utilize the captured data to calculate the appropriate duration of time required for the disinfecting agent to reach disinfection efficacy and dry surface. When this period is complete the device can present a visual and/or auditory alert to indicate the operatory is ready for the next patient. The software application can present an audio and/or visual alert using an application on a mobile device with a progress/countdown monitor.

In some embodiments, the handpiece can include a light or laser to indicate where the disinfecting fluid is being directed.

In some embodiments, the misting/fogging handpiece itself, and/or a handpiece adaptor, and/or a handpiece hose extension can be designed to directly or removably connect into the dental chair handpiece connectors including the following types:
ISO-B 5-Hole Standard Screw-on Connections (1211);
ISO-B 5-Hole Quick-Connect Connections (1215);
ISO-B 5-Hole Quick-Connect Swivel Connections (1216);
ISO-B 5-Hole Economy Lamp Connections (1217);
Kinetic-360 Connections (1221);
ISO-C 6-Pin Connections (1240);
Non-Optic 4-Hole Connections (2430); and
Any new emerging connection types.

In some embodiments, the disinfection fluid container will hold the disinfection fluid and can be used to directly replace the existing water container on the dental chair being swapped out after disinfection is completed. A disinfection fluid selector valve can allow the selection of either disinfection fluid or standard water. A disinfection manifold can allow the chair to have both water bottle and disinfection fluid bottle to be connected to the chair at the same time. The disinfection manifold can include a hypochlorous acid generator which continually generate hypochlorous acid. The disinfection manifold can include a mechanism that automatically purges the hypochlorous on a scheduled basis to assure that hypochlorous always at optimum potency. The disinfection manifold can include a mechanism that automatically purges the hypochlorous from the water lines and flushes them with water after each disinfection session.

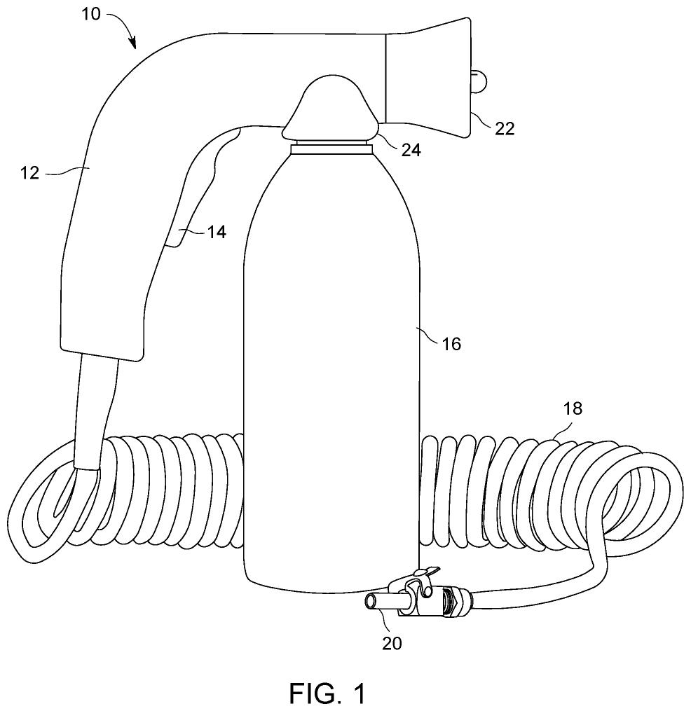

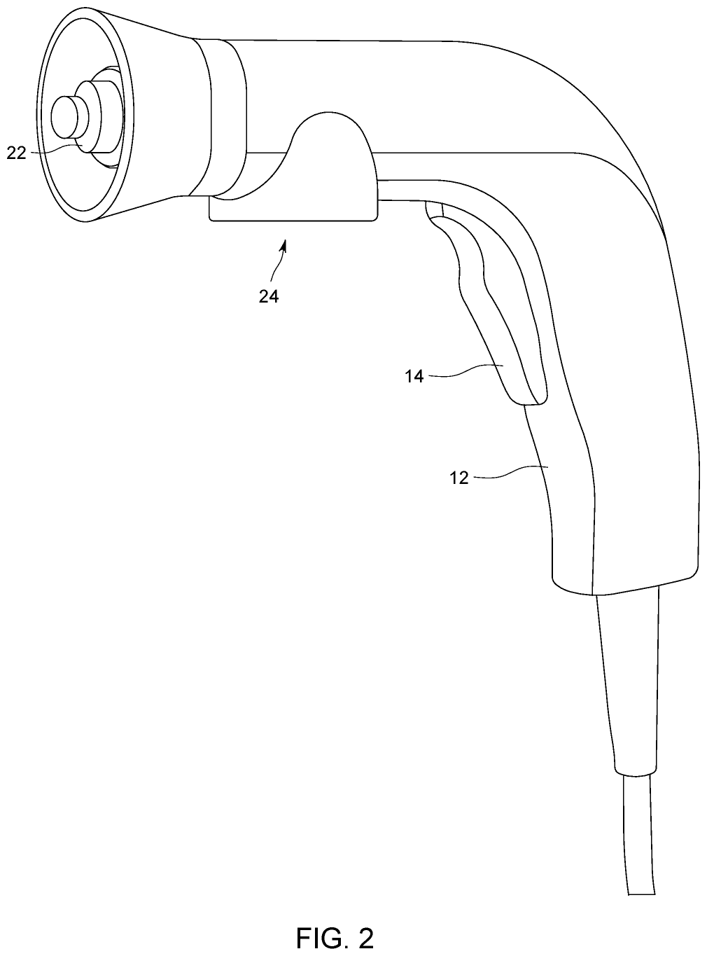

Referring now to FIGS. 1 through 5, a disinfection device 10 can include a disinfection solution bottle 16 (also referred to simply as bottle 16 or container 16) removably attached to a bottle adapter 24 formed in a handpiece 12. The handpiece 12 can include a nozzle 22 for delivering a mist or fog of the solution from the bottle 16. A trigger 14 can be disposed on the handpiece for activating the flow of the solution from the bottle 16 and out of the nozzle 22 as a mist or fog, for example.

An air line 18 can be used to connect the handpiece 12 to a source of pressurized gas, such as pressurized air present in an operatory. Such pressurized air can be, for example, between about 40 to about 100 psi, typically between 60 and 80 psi. The air line 18 can be formed in a coil, as shown, to allow the user to move about the operatory and apply the mist or fog to the entirety of the room. An air line attachment fitting 20 can be used to connect the air line 18 to the source of pressurized gas. The air line attachment fitting 20 can by any fitting allowing removable attachment of the air line to the gas source. Examples of such connections are described above.

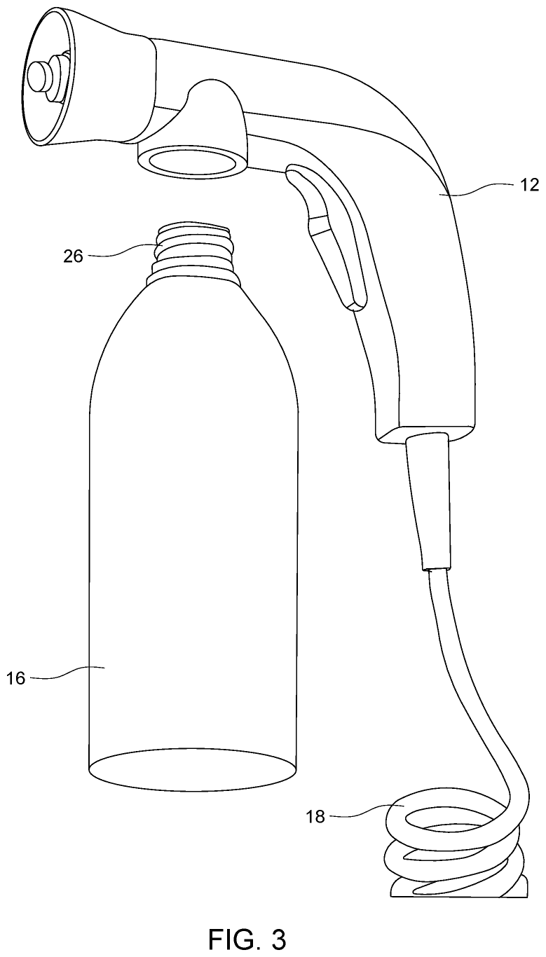

As shown in FIG. 3, the bottle 16 may include a method for removably attaching the bottle 16 to the handpiece 12. For example, the bottle 16 may include threads 26, as shown. In other embodiments, the bottle 16 may include a quick disconnect feature, a press-fit feature, a twist-lock feature, or other similar mechanism to seal the bottle 16 to the handpiece 16. In some embodiments, the bottle 16 may have a seal formed therein, where the seal may be automatically pierced when the bottle 16 is connected to the bottle adapter 24. In other embodiments, the bottle 16 may be opened by the user and attached to the bottle adapter 24 of the handpiece 12.

In some embodiments, the bottle adapter 24 can include a take up hose (not shown) extending downwardly to terminate near a bottom of the bottle 16 when the bottle 16 is connected to the handpiece 12.

The mist or fog may be generated by various methods. For example, the pressure in the pressurized air may provide for the generation of the mist or fog. In some embodiments, an electrostatic nozzle may assist in the generation of the mist or fog.

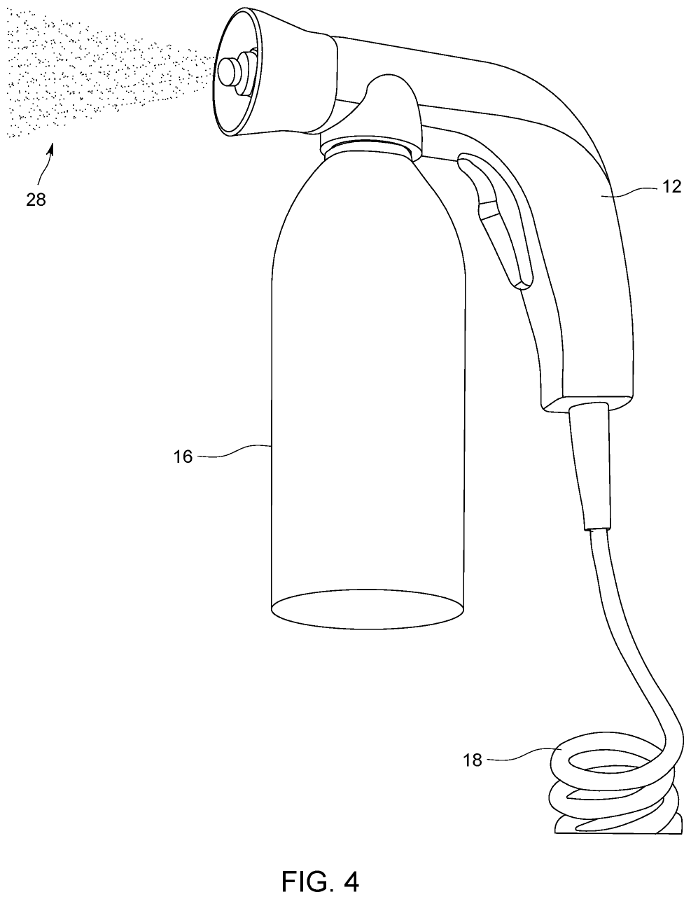

FIG. 4 illustrates the device 10 spraying a fog or mist 28 out of its nozzle. The fog or mist 28 can be, for example, formed of particles of about 10 to 100 microns, where such a fog or mist can expel the nozzle and evenly and uniformly cover a surface at which the spray is directed.

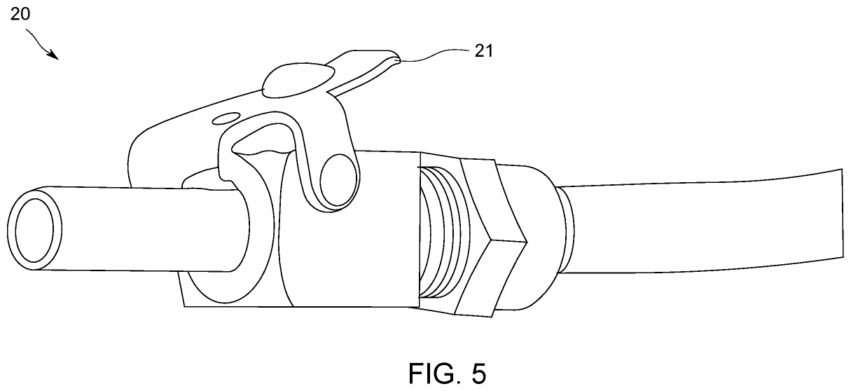

FIG. 5 illustrates one example of the air line attachment fitting 20, where the end can include a lock mechanism 21 that can retain the fitting 20 onto a source of pressurized gas. The fitting 20 is but one example. Other fittings, as may be known in the art, may be used for connecting the air line 18 to the pressurized gas source. In some embodiments, the air line may be permanently connected to the pressurized gas source. Such a configuration may be particularly useful, for example, for wall mount systems, or for those permanently affixed to a furniture element, such as a dental operatory chair. Some such configurations are described in greater detail below.

In some embodiments, a hand-held disinfecting device 60 can include a housing holding a disinfecting solution bottle 66 therein. The housing can include a power source 62, such as a rechargeable battery, and an air pressure generator 64. The device 60 may be stored in a base (not shown) that permits charging of the power source 62. A nozzle 68 may expel a fog or mist in a manner similar to the device 10 described above. Thus, with the hand-held device 60, there is no need for the air line, as the requisite air pressure may be provided within the hand-held unit.

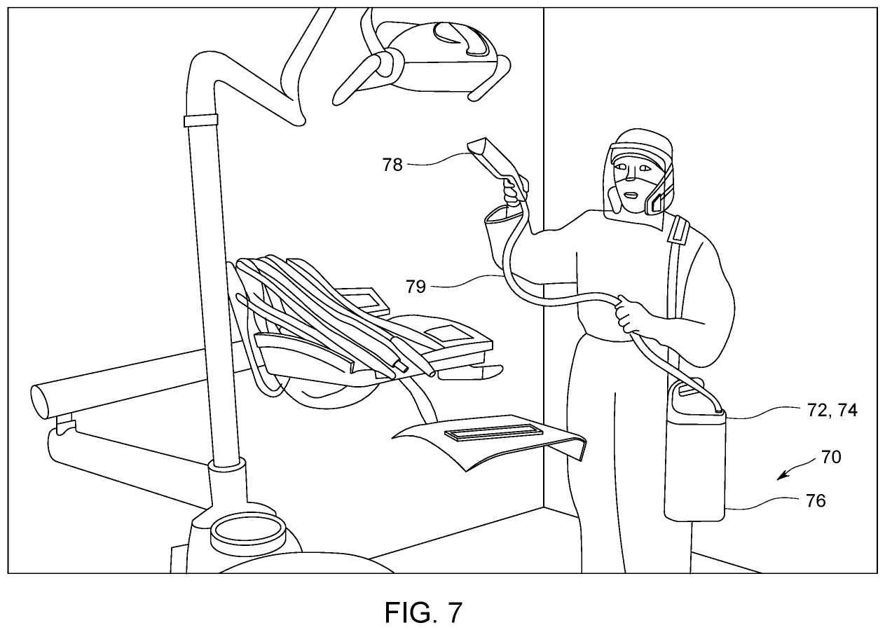

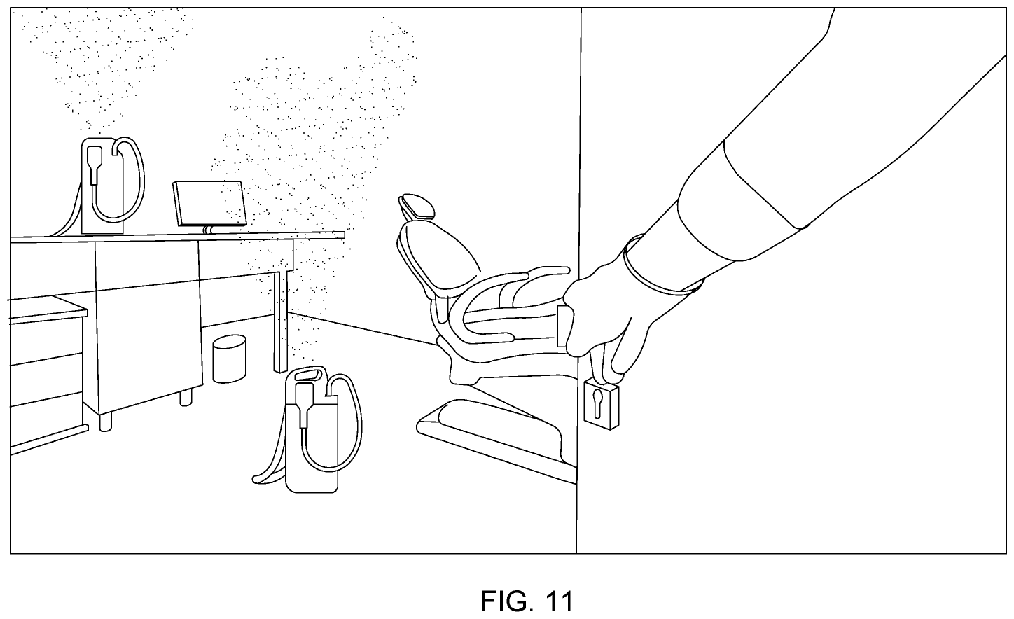

Similarly, as shown in FIG. 7, a portable disinfecting device 70 may be larger than the hand-held device 60 described above and may include a larger bottle 76 for the disinfecting solution. Like the above, the device 70 can include a power source 72, such as a rechargeable battery, and an air pressure generator 74. The nozzle 78 may connect to the base unit via a tubing 79, so that the device 70 may be worn, for example, over the user's shoulder as shown, while the user moves the nozzle 78 about to deliver the fog or mist. The portable device 70 may be used, as shown in FIG. 11, by placing the device in a room and letting the device deliver a mist or fog into the room automatically, without requiring the user to direct the nozzle. One or more of the devices 70 may be used to adequately disinfect an entire room. In some embodiments, the device may be mobile to move about the room as it sprays or may turn in a circle to deliver the fog of mist over a broad area.

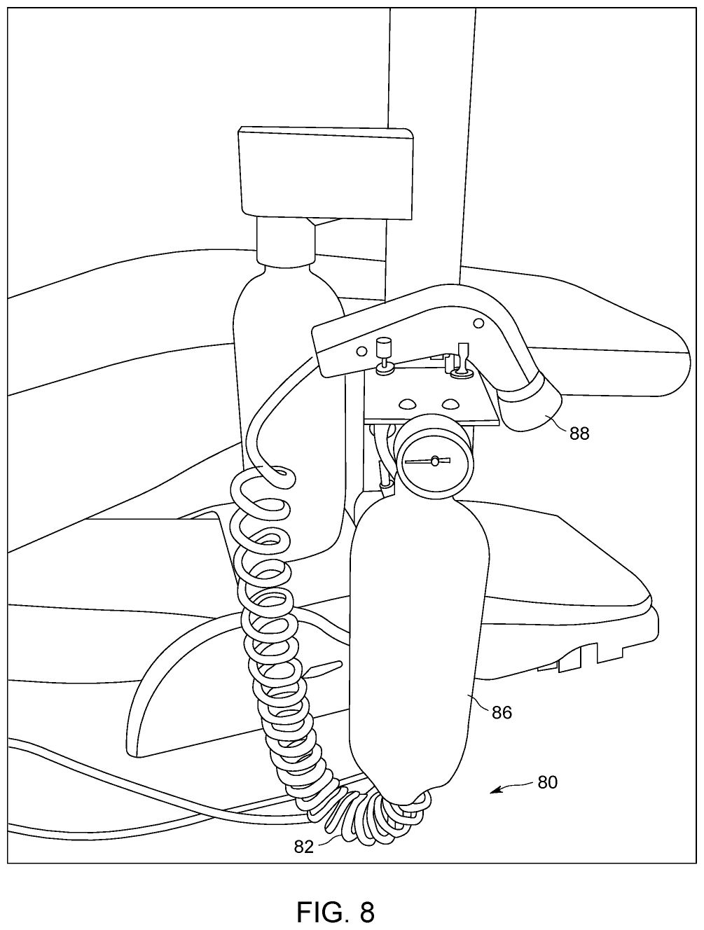

In other embodiments, a dedicated system 80 can be as shown in FIG. 8. Such a system can attach to an existing post in a dental operatory. The device 80 can include pressure gauge to measure air pressure and an adjustment knob to adjust the air pressure of the system. The device 80 can include an on/off switch and an air pressure release to permit change-out of the solution containers 86. The device may be centrally mounted in a room for disinfection throughout the room or may be mounted near entries/exits of the room to permit people to self-disinfect when entering or leaving a room. The solution container 86 can be, for example, a screw-on container for easy change-out when empty, when expired, when the concentration is too low, or the like.

A tubing 82 can connect the base portion to a nozzle 88. In some embodiments, the nozzle 88 may include a trigger to control the flow of the fog of mist. In other embodiments, the nozzle 88 can simply deliver the fog or mist for the entire time the system is turned on via the switch on the base portion.

In some embodiments, the device can be a mobile device, disposed on a cart with a power supply, such as a battery. The mobile system can also be hand-held, with a smaller, rechargeable battery, as described above.

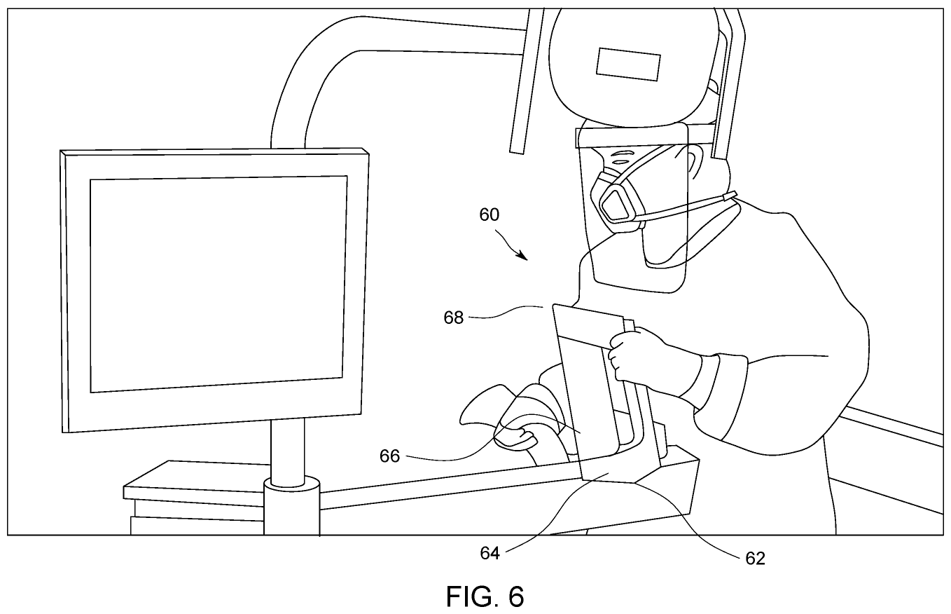

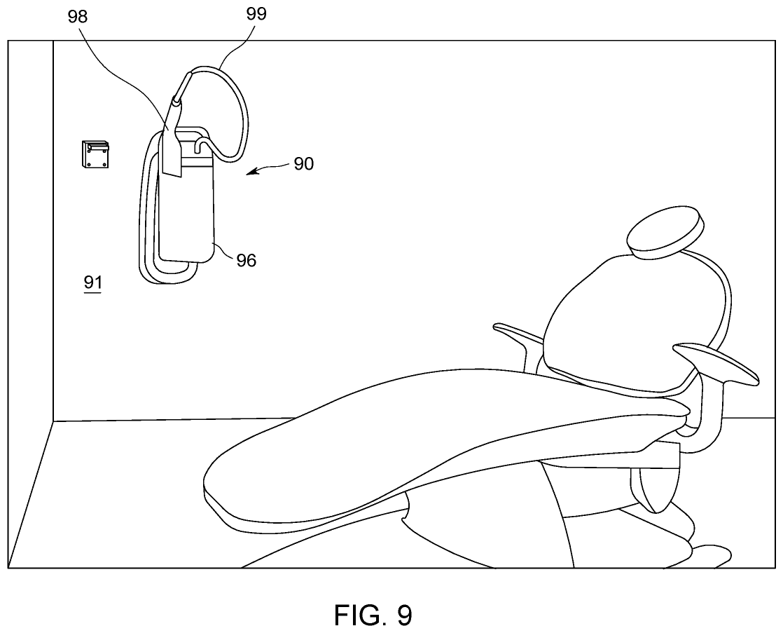

In some embodiments, a device 90 can mount on a wall 91, as shown in FIG. 9, in a wall cavity or the like. The device 90 can be similar to other devices discussed above. The device 90 can include a disinfecting solution bottle 96, a nozzle 98, and tubing 99 connecting the nozzle 98 with the bottle 96. The device 90 can be fixed to the wall, where the user uses the tubing 99 to move the nozzle 98 about the room to deliver the mist or fog. In this embodiment, pressurized air may be provided from the building itself (such as the device of FIG. 1, that connects to a pressurized gas source in the operatory), or pressurized gas may be generated by a pressurized gas generator disposed in the device itself. In other embodiments, the device 90 may be removable from the wall, operating similar to the rechargeable devices 60, 70 described with respect to FIGS. 6 and 7, for example.

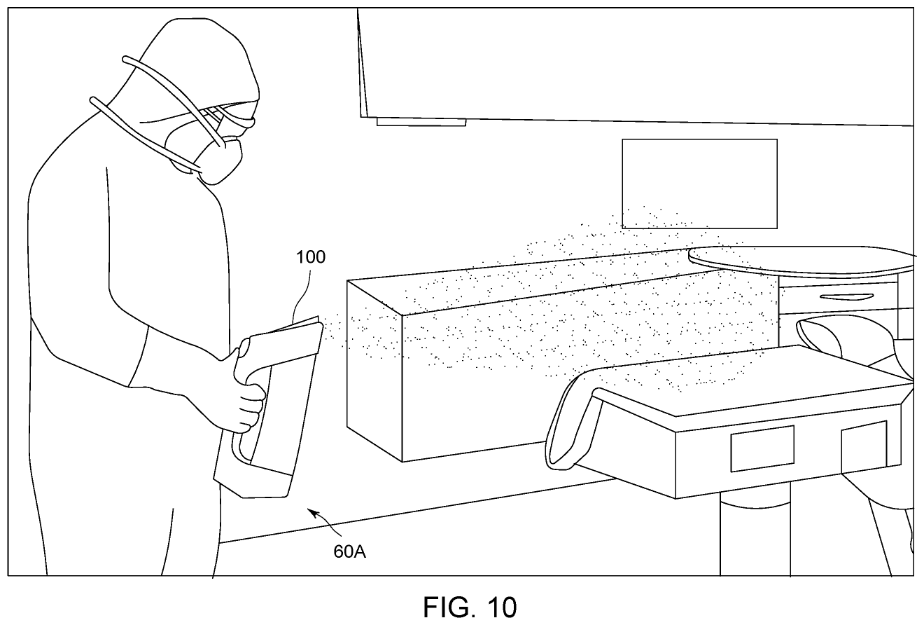

In some embodiments, as shown in FIG. 10, a hand-held device 60A can include a light 100. The light 100 may automatically turn on to shine on the mist or fog as it is expelled from the device, or the light may be manually operated by the user with a separate power button, for example. In some embodiments, the light 100 may pop up out of the housing, as shown in FIG. 10. While FIG. 10 illustrates the light 100 on the hand-held device 60A, it should be understood that a light may be used on the device 10 of FIG. 1, the device 70 of FIG. 7 or other similar devices.

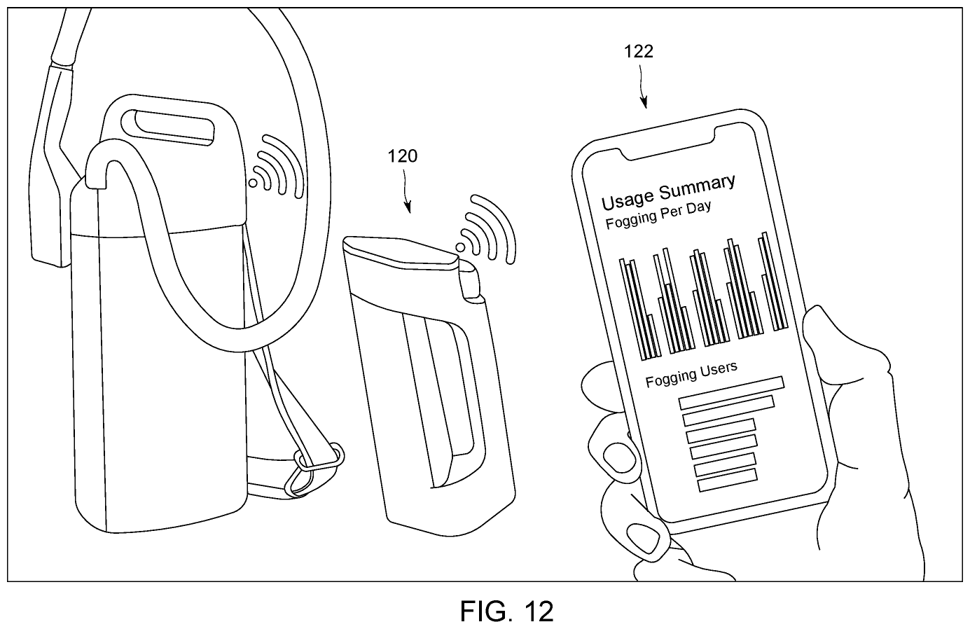

As shown in FIG. 12, a device 120 may include a wireless transmitter (not shown), such as a Wi-Fi® or Bluetooth® transmitter that can send and receive data. A mobile computing device 122 can include software configured to receive and send data to the device 120. The software can provide data to the user, including amount of fogging performed each day for each device, the users who perform the fogging, the amount of fogging in each room, the days and times of fogging in each room, and the like. The software can further include a timer that alerts the user, after completion of fogging, when occupancy can occur. The software can further monitor battery charge (if applicable in the device), disinfecting solution levels, disinfecting solution concentrations, and the like. For these systems, the devices can include related sensors, such as solution level sensors, hypochlorous concentration sensors, battery charge sensors, user sensors (such as thumb print detection), and the like, which can send data to the mobile computing device 122. In some embodiments, these sensors can provide indications on the device itself. For example, the device may include indicators to show low battery (or battery level), solution level, solution concentration, or the like.

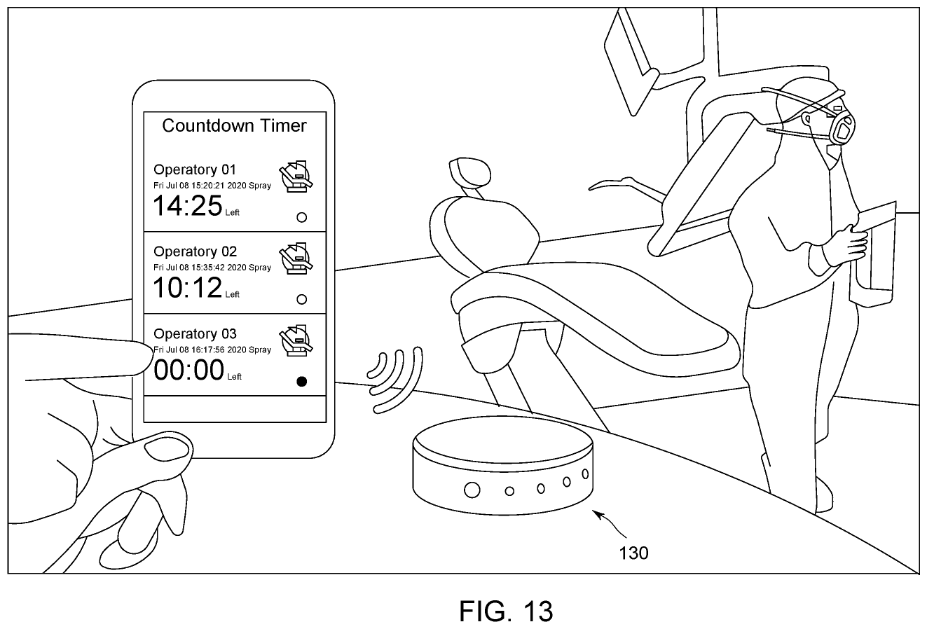

Referring now to FIG. 13, in some embodiments, a timer device 130 can provide a visual and/or audio indicators for various timing functions. For example, the timer device 130 can provide users with an indication of how long they have been actively fogging in a room. The timer device 130 can further provide users with an indication of a predetermined period of time after fogging is completed, where the disinfecting is complete and users may occupy the room. Such a timer may further be automatically adjusted for factors such as hypochlorous concentration in the mist or fog, temperature, humidity, or the like.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

Claim elements and steps herein may have been numbered and/or lettered solely as an aid in readability and understanding. Any such numbering and lettering in itself is not intended to and should not be taken to indicate the ordering of elements and/or steps in the claims.

Many alterations and modifications may be made by those having ordinary skill in the art without departing from the spirit and scope of the invention. Therefore, it must be understood that the illustrated embodiments have been set forth only for the purposes of examples and that they should not be taken as limiting the invention as defined by the following claims. For example, notwithstanding the fact that the elements of a claim are set forth below in a certain combination, it must be expressly understood that the invention includes other combinations of fewer, more or different ones of the disclosed elements.

The words used in this specification to describe the invention and its various embodiments are to be understood not only in the sense of their commonly defined meanings, but to include by special definition in this specification the generic structure, material or acts of which they represent a single species.

The definitions of the words or elements of the following claims are, therefore, defined in this specification to not only include the combination of elements which are literally set forth. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements. The claims are thus to be understood to include what is specifically illustrated and described above, what is conceptually equivalent, what can be obviously substituted and also what incorporates the essential idea of the invention.

What is claimed is:

1. A device for disinfecting surfaces comprising:
an air line;
a connector at a first end of the air line, the connector operable to connect the air line to a pressurized gas source;
a handpiece at a second end of the air line;
a container configured to contain a disinfectant solution, the container removably attached from the handpiece;
a nozzle configured to expel a mist or fog away from the handpiece; and
a trigger mechanism on the handpiece operable to control flow of the mist or fog of the disinfectant solution out of the nozzle.

2. The device of claim 1, wherein the connector is configured to connect to a pressurized air source in an operatory.

3. The device of claim 1, wherein the connector is removably attached to the pressurized gas source.

4. The device of claim 1, wherein the disinfectant solution is a hypochlorous acid solution.

5. The device of claim 1, further comprising a light operable to illuminate in a direction that the mist or fog exits the nozzle.

6. The device of claim 1, further comprising a remote computing device having non-transitory computer readable storage medium tangibly embodying a computer readable program code having computer readable instructions that, when executed, causes the computer device to carry out a method, the method comprising:
receiving data from the device; and
displaying use data for the device, wherein the use data includes at least one of user identification, duration of use, date and time of use, wait time between termination of use and occupancy of a room where used, or location data of use.

7. The device of claim 1, further comprising a timer mechanism, the timer mechanism configured to provide an indication when a room treated the fog or mist from the device is ready for occupancy.

8. The device of claim 7, wherein the timer mechanism receives data from the device to begin a countdown to providing the indication.

9. A device for disinfecting surfaces comprising:
a base unit operable to receive pressurized gas from a pressurized gas source;
a handpiece connected to the base unit via a tubing;
a container configured to contain a disinfectant solution, the container removably attached from the base unit;
a nozzle disposed at an end of the handpiece and configured to expel a mist or fog away from the handpiece; and
a trigger mechanism operable to control flow of the mist or fog of the disinfectant solution out of the nozzle.

10. The device of claim 9, wherein the base unit and handpiece are integral.

11. The device of claim 10, further comprising a power source within the device for powering a pressurized gas generator for generating pressurized gas at the pressurized gas source, wherein the pressurized gas source is integrated within the device.

12. The device of claim 11, wherein the device is hand-held and cordless.

13. The device of claim 9, wherein the device includes a hand-held nozzle remote from the base unit, the hand-held nozzle connected to the base unit by the tubing.

14. The device of claim 9, wherein the device is operable to generate the fog or mist without user interaction with the device.

15. A method for disinfecting a room, comprising:
drawing a disinfecting solution from a container;
forming a mist or fog and delivering the mist or fog using pressurized gas from a pressurized gas source;
expelling the mist or fog out from a nozzle of the device; and
allowing the mist or fog to contact surfaces of the room.

16. The method of claim 15, wherein:
the container is removably attached to a handpiece having the nozzle formed therein; and
the handpiece removably connects to the pressurized gas source with an air line.

17. The method of claim 15, wherein the disinfecting solution is a hypochlorous acid solution.

18. The method of claim 15, wherein:
the nozzle is integral with a cordless handheld unit;
the container is removably attached to the cordless handheld unit;
a power supply within the cordless handheld unit provides power to the pressurized gas source; and
the pressurized gas source is disposed within the cordless handheld unit.

19. The method of claim 15, wherein:
the nozzle is attached to a base unit via tubing; and
the container removably attaches to the base unit.

20. The method of claim 19, wherein:
a power supply is disposed within the base unit to provide power to the pressurized gas source; and
the pressurized gas source is disposed within the base unit.
